(19) United States
(12) Patent Application Publication
(10) Pub. No.: US 2010/0016712 A1
(43) Pub. Date: Jan. 21, 2010

(54) Method and Device for Visually Assisting a Catheter Application

(76) Inventors: Meir Bartal, Zichron Yaakov (IL); Jan Boese, Eckental (DE); Assaf Govari, Haifa (IL); Matthias John, Nurnberg (DE); Assaf Preiss, Shimshit Israel (IL); Norbert Rahn, Forchheim (DE)

Correspondence Address: SIEMENS CORPORATION;INTELLECTUAL PROPERTY DEPARTMENT, 170 WOOD AVENUE SOUTH, ISELIN, NJ, 08830, US

(21) Appl. No.: 12/518,172

(22) PCT Filed: Feb. 26, 2008
(86) PCT No.: PCT/EP2008/052295

§ 371 Date: Jun. 8, 2009

(30) Foreign Application Priority Data
Feb. 27, 2007 (DE) 10 2007 009 764.8

(51) Int. Cl. A61B 6/12; A61B 5/055; G06K 9/00

(52) U.S. Cl. 600/425; 382/132

(57) ABSTRACT

A method and a device for visually assisting a catheter application on the heart of a patient using an image of the patient obtained by a C-arm X-ray device and using electroanatomical mapping data of the patient obtained by an electromagnetic position detection system and mapping system. The C-arm X-ray device and the electromagnetic position detection system and mapping system are calibrated in relation to each other, by determining a co-ordinate transformation between a co-ordinate system assigned to the C-arm x-ray device and/or a co-ordinate system assigned to the image generated by the C-arm X-ray device and a co-ordinate system assigned to the electromagnetic position detection system and mapping system. The position of the patient is determined during the detection of the image and/or during the detection of the electroanatomic mapping-data and is at least indirectly assigned to the image and/or the electroanatomic mapping-data.

### CROSS REFERENCE TO RELATED APPLICATIONS

This application is the US National Stage of International Application No. PCT/EP2008/052295, filed Feb. 26, 2008 and claims the benefit thereof. The International Application claims the benefits of German application No. 10 2007 009 764.8 filed Feb. 27, 2007, both of the applications are incorporated by reference herein in their entirety.

### FIELD OF THE INVENTION

The invention relates to a method and to a device for visually assisting a catheter application on the heart of a patient using at least one image of the patient obtained by means of a C-arm X-ray device and using electroanatomical mapping data relating to the patient, obtained by means of an electromagnetic position-detection and mapping system.

### BACKGROUND OF THE INVENTION

In various fields where medical technology finds application, it is customary nowadays for a medical instrument, a punction needle or a catheter, for example, to be introduced into a patient in a targeted manner with the aid of image information made available by imaging devices, in order to examine or treat the patient or a tissue or organ of the patient with the instrument.

Thus, for example, cardiac dysrhythmias in a patient are treated using what is known as an ablation, in which an ablation catheter is introduced through veins or arteries into one of the chambers of the patient's heart, under X-ray monitoring, based on X-ray images, for example, and the tissue responsible for the dysrhythmias is destroyed by high frequency current. The prerequisite for carrying out a successful catheter ablation is the precise locating of the cause of the dysrhythmias in the chamber of the heart. This is achieved by means of an electrophysiological investigation in which electrical potentials are detected in a locally resolved manner using a mapping catheter introduced into the chamber of the heart. From this electrophysiological investigation, known as electroanatomical mapping, 3D mapping data, for example, which can be visualized on a monitor, are thus obtained. The mapping function and the ablation function are frequently combined in one catheter so that the mapping catheter is also an ablation catheter at the same time.

A known electroanatomical 3D mapping method, such as is possible, for example, using the CARTO-System from the company Biosense Webster Inc., USA, is based on electromagnetic principles. Three different low-intensity electromagnetic alternating fields are generally established using transmitters arranged under a patient supporting device. By means of electromagnetic sensors incorporated in the catheter tip of the mapping catheter, it is then possible to measure the changes in voltage within the electromagnetic alternating fields induced by catheter movements and to calculate the position of the mapping catheter at any time with the aid of mathematical algorithms. Through point-by-point mapping of the endocardial contour of a chamber of the heart with the mapping catheter and simultaneous detection of the electrical signals from the sensors, 3D mapping data is obtained or an electroanatomical three-dimensional map is produced, in which the electrical signals are reproduced in a color-coded manner.

The guidance of the ablation catheter can thus be achieved not only with the aid of the aforementioned X-ray images, but also using the electroanatomical mapping data, which can be generated in real time using for example, the aforementioned CARTO-System from the company Biosense Webster Inc., USA and displayed on a monitor. The fact is that the X-ray images do not in fact show in detail the anatomy of the patient or, in particular, the anatomy of the patient's heart. A 3D view of anatomical details of the heart could increase the precision relative to the morphology of the heart tissue during an ablation procedure, speed up the ablation procedure and lead to a reduction in the X-ray dose applied to a patient during an ablation.

Electrophysiologists welcome the opportunity, particularly in complex cases, of being able to carry out the ablation using a combination of electrophysiological and morphological criteria. It would therefore be helpful for electrophysiologists to have at their disposal a combined visualization of 3D image data obtained using an image-generating device and electroanatomical 3D mapping data.

DE 103 40 544 A1 and DE 103 40 546 A1 disclose, for example, methods for visually assisting a catheter application, in which 3D image data of an area of the patient that is to be treated is acquired by a method of tomographic 3D image generation before the catheter application is carried out, a 3D surface contour of objects in the area to be treated being extracted from the 3D image data by segmentation and electroanatomical 3D mapping data that are subsequently provided and 3D image data forming the 3D surface contour being assigned to each other in the correct position and dimensions and being visualized during the catheter application procedure, for example, such that they are superimposed on one another. This superimposition of 3D image data relating to the patient obtained before the catheter application, by means of computer tomography or magnetic resonance tomography, for example, and electroanatomical 3D mapping data relating to the patient sometimes requires a time-consuming and error-prone marker-based or surface-based registration of the data with one another. Here, errors in registration can have a negative effect on the quality and reliability of images that comprise merged data. In this method, the precision of registration therefore also depends on the number of surface points that are obtained using the mapping system.

### SUMMARY OF THE INVENTION

The object underlying the invention is therefore to provide a method and a device of the type mentioned in the introduction, such that the combined use of image data and mapping data is simplified.

This object is achieved according to the invention by a method and a device for visually assisting a catheter application on the heart of a patient using at least one image of the patient obtained by means of a C-arm X-ray device and using electroanatomical mapping data relating to the patient, obtained by means of an electromagnetic position-detection and mapping system. The C-arm X-ray device and the electromagnetic position-detection and mapping system are calibrated in relation to each other, by determining a coordinate transformation between a coordinate system assigned to the C-arm X-ray device and/or a coordinate system that is assigned to at least one image generated by the C-arm X-ray device and a coordinate system assigned to the electromagnetic position-detection and mapping system. The position of the patient is determined during the acquisition of the image and/or during the acquisition of the electroanatomical mapping data and is at least indirectly assigned to the image and/or to the electroanatomical mapping-data.

By calibrating the C-arm X-ray device and the electromagnetic position-detection and mapping system in relation to each other, registration of image data relating to an image generated by the C-arm X-ray device and mapping data generated by the electromagnetic position-detection and mapping system during a catheter application is no longer required, since the transformation relationship for the data from the two systems or devices is now known. Consequently, assuming that the patient has not changed position between and after the acquisition of data using the two systems or devices, it is possible to merge together or superimpose on one another image data from images of the patient obtained before or during a catheter application using the C-arm X-ray device, whether comprising 2D images or 3D images, and mapping data obtained before or during the catheter application, whether it is 2D or 3D mapping data, without having to carry out a time-consuming or error-prone registration of the data. Moreover, as a result of the fact that the position of the patient is determined during the acquisition of the image and/or the acquisition of the electroanatomical mapping data and is assigned at least indirectly to the image and/or to the electroanatomical mapping data, it is additionally possible accordingly to take into account, in the image processing, changes in the position of the patient after the acquisition of the image using the C-arm X-ray device and the acquisition of the electroanatomical mapping data.

On the C-arm X-ray device, which is provided for angiographic applications, for example, an X-ray source and X-ray receiver are arranged opposite each other on a C-arm, which can be adjusted to record 2D projections from different projection directions around the patient. A volume data set can be reconstructed from a series of 2D projections recorded with the C-arm X-ray device at projection directions that differ from each other. On the basis of the known dimensions of the C-arm X-ray device and the known projection geometries of the 2D projections, the transformation relationship between a coordinate system assigned to the C-arm X-ray device and a coordinate system assigned to the volume data set or to a 3D image generated from the volume data set is also known.

According to a variant of the invention, a patient supporting device is assigned to the C-arm X-ray device in a defined manner, that is, the C-arm X-ray device and patient supporting device are arranged relative to each other in a known manner.

According to a further variant of the invention, the electromagnetic position-detection and mapping system comprises at least one transmitter to generate an electromagnetic alternating field and a catheter with at least one sensor. Usually the electromagnetic position-detection and mapping system comprises a plurality of transmitters, for example, three transmitters, to generate three different alternating fields and the catheter comprises three sensors, such that the position of the catheter can be determined in a coordinate system assigned to the electromagnetic position-detection and mapping system with the aid of the three sensors incorporated in the catheter.

To determine the coordinate transformation between a coordinate system assigned to the C-arm X-ray device and/or a coordinate system assigned to at least one image generated with the C-arm X-ray device and a coordinate system assigned to the electromagnetic position-detection and mapping system, according to a variant of the invention the transmitter is arranged in a defined manner on the C-arm X-ray device or on the patient supporting device. According to an embodiment of the invention, the transmitter is arranged in a defined manner on the C-arm of the C-arm X-ray device. In this way, a firm relationship can be established between the C-arm X-ray device and the electromagnetic position-detection and mapping system.

According to an embodiment of the invention, the transmitter is detachable from the C-arm X-ray device or from the patient supporting device. Therefore, if the transmitter interferes, for example, with the taking of images of the patient using the C-arm X-ray device, then it can be detached from the C-arm X-ray device or the patient supporting device while images are being taken by the C-arm X-ray device and then be arranged again in the defined position on the C-arm X-ray device or on the patient supporting device.

According to a different variant of the invention, at least one positioning and orientation sensor of the electromagnetic position-detection and mapping system is arranged on or in the patient supporting device in a defined manner. In this case, on the basis of the known relationship between the C-arm X-ray device and the patient supporting device and on the basis of the known design arrangement of the at least one positioning and orientation sensor on or in the patient supporting device, the transformation relationship between the coordinate system assigned to the C-arm X-ray device and thus also between the coordinate system assigned to an image recorded using the C-arm X-ray device and the coordinate system assigned to the electromagnetic position-detection and mapping system can be determined by detecting the positioning and orientation sensor with the electromagnetic position-detection and mapping system.

According to an embodiment of the invention, the at least one positioning and orientation sensor of the electromagnetic position-detection and mapping system is detachable from the patient supporting device. Preferably, the positioning and orientation sensor or the positioning and orientation sensors is/are arranged in a module that is detachable from the patient supporting device or in a plurality of modules that are detachable from the patient supporting device, said modules being arranged relative to each other on the patient supporting device in a defined manner.

According to a variant of the invention, the determination of the coordinate transformation between a coordinate system assigned to the C-arm X-ray device and/or a coordinate system assigned to at least one image generated with the C-arm X-ray device and a coordinate system assigned to the electromagnetic position-detection and mapping system is achieved by means of at least one marker, which is depicted in an image that is generated and is detectable using the electromagnetic position-detection and mapping system. Usually a plurality of markers, preferably at least three markers, are used to determine the coordinate transformation, the coordinates of each of the individual markers being determined in the respective coordinate systems and the coordinate transformation between the coordinate systems being ascertained using the coordinates that have been determined for the respective markers in the coordinate systems.

The marker or markers are X-ray positive markers, for example small metal balls, which are depicted in at least two X-ray projections taken at projection angles that differ from each other or in a 3D image. According to embodiments of the invention, the images of the markers can be located in the projection images or the 3D image either manually through a graphic user interface or automatically using a method of pattern recognition, such that the marker coordinates required for the determination of the coordinate transformation can be determined in the coordinate system assigned to the projection images or the 3D image by back projection.

In order to determine the coordinates of the markers in the coordinate system assigned to the electromagnetic position-detection and mapping system, a position sensor of the electromagnetic position-detection and mapping system is used, with which the respective markers are touched.

According to a variant of the invention, at least one marker is an X-ray positive position sensor of the electromagnetic position-detection and mapping system. Since the marker itself is a position sensor of the electromagnetic position-detection and mapping system, said marker does not specifically have to be touched by another position sensor in order for the coordinates thereof in the coordinate system assigned to the electromagnetic position-detection and mapping system to be detected and determined, but can be detected directly and automatically by the electromagnetic position-detection and mapping system. Here, the marker or the X-ray positive position sensor can be a catheter tip of a catheter from the electromagnetic position-detection and mapping system.

According to an embodiment of the invention, a phantom comprising at least one marker is provided for the determination of the coordinate transformation, said phantom being arranged in an appropriate manner on the patient supporting device relative to the C-arm X-ray device and the electromagnetic position-detection and mapping system in order to determine the coordinate transformation.

According to a variant of the invention, at least one marker of said phantom may be a position sensor of the electromagnetic position-detection and mapping system such that, as already mentioned, the marker does not specifically have to be touched by a position sensor, but can be detected directly and automatically by the electromagnetic position-detection and mapping system.

According to an embodiment of the invention, all the markers of the phantom are position sensors of the electromagnetic position-detection and mapping system such that the markers of the phantom can be detected directly and automatically by the electromagnetic position-detection and mapping system and the coordinates thereof can be determined in the coordinate system assigned to the electromagnetic position-detection and mapping system.

According to a further variant of the invention, the phantom comprises, at least one, preferably a plurality of further markers that are not position sensors, in addition to the position sensor of the electromagnetic position-detection and mapping system, the positions or coordinates of the further markers of the phantom being known relative to the at least one position sensor of the phantom. In this case it is sufficient to detect the position sensor automatically, using the electromagnetic position-detection and mapping system and to determine the coordinates thereof. The coordinates of the further markers in the coordinate system assigned to the electromagnetic position-detection and mapping system are then obtained from the known coordinates of the further markers relative to the position sensor.

According to an embodiment of the invention, the phantom that comprises markers and the electromagnetic position-detection and mapping system are arranged in a defined manner relative to each other, such that the coordinate transformation between a coordinate system assigned to the phantom and a coordinate system assigned to the electromagnetic position-detection and mapping system is known. In this case, therefore, the coordinates of the markers of the phantom do not themselves have to be determined in the coordinate system assigned to the electromagnetic position-detection and mapping system. In fact, in order to calculate the coordinate transformation between the coordinate system assigned to the C-arm X-ray device or the coordinate system assigned to an image obtained using the C-arm X-ray device and the coordinate system assigned to the electromagnetic position-detection and mapping system, it is only the coordinates of the markers of the phantom that have to be determined in the coordinate system assigned to the image obtained using the C-arm X-ray device, in order to be able to calculate the coordinate transformation.

The methods described hitherto for the determination of the coordinate transformation between the coordinate system assigned to the C-arm X-ray device or the coordinate system assigned to the image acquired using the C-arm X-ray device and the coordinate system assigned to the electromagnetic position-detection and mapping system are what are known as offline methods, in which a patient who needs to be examined is not present.

The determination of the coordinate transformation may also be achieved online, however, that is, in the presence of the patient. According to this variant of the invention, the patient or a part or an organ of the patient is provided with markers, preferably with X-ray positive markers, the images whereof are located in an image obtained using the C-arm X-ray device and which can be detected using the electromagnetic position-detection and mapping system. Next, the coordinates of the markers in the coordinate systems assigned to the image and the coordinates of the markers in the coordinate system assigned to the electromagnetic position-detection and mapping system are again determined and on this basis, the coordinate transformation between the coordinate system assigned to the C-arm X-ray device or to the image generated using the C-arm X-ray device and the coordinate system assigned to the electromagnetic position-detection and mapping system is determined.

According to a variant of the invention, the position of the patient is determined continuously or intermittently during the catheter application. In this way, the position of the patient is constantly known, so that changes in the position of the patient since the acquisition of the image using the C-arm X-ray device and/or the acquisition of the electroanatomical mapping data is recorded and can be taken into account accordingly in the image processing and use of the image data.

According to an embodiment of the invention, the image data for the image obtained using the C-arm X-ray device and the electroanatomical mapping data is merged together or superimposed during the acquisition of the image and of the electroanatomical mapping data, or superimposed on one another, taking into account the position of the patient (P). Therefore, if for example, the patient changes position between the acquisition of the image data for the image using the C-arm X-ray device and the acquisition of the electroanatomical mapping data, then on the basis of the change in the position of the patient that has been recorded and determined, the previously determined coordinate transformation may be adapted accordingly, such that the electroanatomical mapping data and the image data for the image that was generated previously can continue to be merged together or superimposed with positional and locational precision.

According to a further variant of the invention, at least part of a catheter that is detectable using the electromagnetic position-detection and mapping system or of an instrument that is detectable using the electromagnetic position-detection and mapping system is blended into the image of the patient and/or the electroanatomical mapping data, taking into account the position of the patient. In this case, too, an adjustment of the coordinate transformation is made, taking into account the change in the position of the patient, such that an image of the catheter or of another instrument can be blended with positional and locational precision into an image that has been taken of the patient that can also comprise electroanatomical mapping data that has been acquired beforehand or at the same time during the navigation of the catheter or of the instrument.

According to an embodiment of the invention, the patient is provided with at least one reference sensor of the electromagnetic position-detection and mapping system for detecting the position of the patient, with the result that the position of the patient can be determined in the coordinate system assigned to the electromagnetic position-detection and mapping system during the determination of an image using the C-arm X-ray device, during the determination of the electroanatomical mapping data and during the catheter application using the electromagnetic position-detection and mapping system.

According to a variant of the invention, the positions or coordinates determined using the electromagnetic position-detection and mapping system are determined relative to the reference sensor of the patient. In this case there is a direct registration between the coordinate system assigned to an image and a coordinate system assigned to the patient through a reference sensor.

According to an embodiment of the invention, a time synchronization is achieved between the acquisition of an image of the patient using the C-arm X-ray device and the position of the patient, the time of acquisition being assigned to an image that has been taken and the position of the patient being determined over time, such that the position of the patient at the time of acquisition of the image can be determined by comparing the times and the image acquired can be assigned to this position of the patient.

According to a different embodiment of the invention, the C-arm X-ray device assigns an identifier to an image that has been taken of the patient at the time of acquisition of the image and transmits the identifier to the electromagnetic position-detection and mapping system at the time of acquisition of the image. The electromagnetic position-detection and mapping system then stores the position of the patient, including the identifier, at the time of acquisition of the image.

According to a further variant of the invention, the C-arm X-ray device retrieves the position of the patient from the electromagnetic position-detection and mapping system at the time an image of the patient is acquired and assigns it to the image that has been acquired.

These variants of the invention make it possible in each case to register a change in the position of the patient relative to a position the patient assumed earlier, in which an image was generated, and to take into account the change of position in the superimposition or merging of image data and mapping data or when blending in a catheter or an instrument into the image data recorded beforehand.

### BRIEF DESCRIPTION OF THE DRAWINGS

An embodiment of the invention is shown in the attached schematic drawings. The drawings show:

FIG. 1 a device comprising an electromagnetic position-detection and mapping system, a C-arm X-ray device and computing devices, together with a patient supporting device and

FIG. 2 the device from FIG. 1 comprising a phantom that is arranged on the patient supporting device.

### DETAILED DESCRIPTION OF THE INVENTION

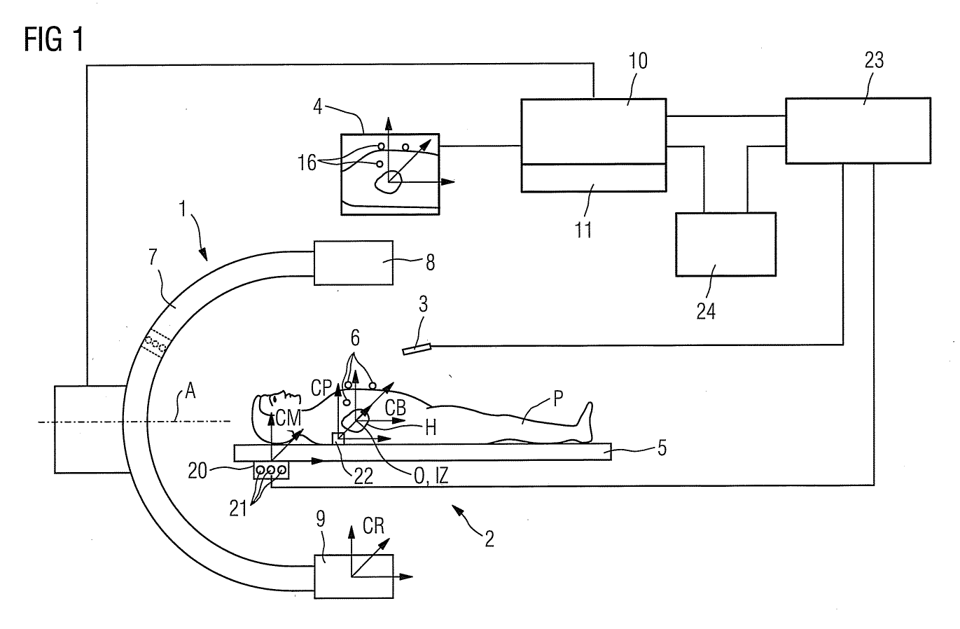

FIG. 1 shows a device comprising a C-arm X-ray device 1 and an electromagnetic position-detection and mapping system 2, which device is used for visually assisting a catheter application on a living being, for example, a catheter ablation on the heart H of the patient P. In the case of the present embodiment, a catheter 3 of the electromagnetic position-detection and mapping system 2, which in the present case is both a mapping catheter and an ablation catheter, is to be guided through veins or arteries into the chamber of the heart H of the patient P, supported by at least one image of the heart H of the patient P obtained using the C-arm X-ray device 1 and supported by mapping data of the heart H of the patient P obtained using the electromagnetic position-detection and mapping system 2, in order to make it possible to carry out an ablation procedure there to treat cardiac dysrhythmias. For this purpose, image data for an image obtained using the C-arm X-ray device 1 and electroanatomical mapping data obtained using the electromagnetic position-detection and mapping system 2 are to be merged together or superimposed on one another in order to simplify the intervention on the patient P. Moreover, the position of the catheter 3 that has been introduced into the patient P is to be blended into the image data and mapping data that has been merged together or superimposed on one another and is displayed on a monitor 4. In order to be able to merge together or superimpose on one another the image data for the image obtained using the C-arm X-ray device 1 and the electroanatomical mapping data obtained using the electromagnetic position-detection and mapping system, or to be able to blend an image of the catheter 3 into the merged or superimposed data with locational and positional precision, the C-arm X-ray device 1 and the electromagnetic position-detection and mapping system 2 are calibrated relative to each other.

For this purpose, the patient P is supported on a patient supporting device 5 assigned to the C-arm X-ray device 1 in a defined manner. Assignment of the C-arm X-ray device 1 and the patient supporting device 5 in a defined manner is understood to mean that the spatial arrangement of the two devices is known even when the devices or parts of the devices are moved relative to each other. The patient P is preferably provided with at least 3 X-ray positive markers 6 in the region of their heart H. The markers 6 are arranged on the patient P such that said markers are depicted in X-ray images of the heart H of the patient P that can be taken using the C-arm X-ray device 1 without obscuring details of the heart H, and such that these details can be detected by the electromagnetic position-detection and mapping system 2. In this way, a coordinate transformation between a coordinate system CR assigned to the C-arm X-ray device 1 or between a coordinate system CB that is assigned to an image generated by the C-arm X-ray device 1 and a coordinate system CM assigned to the electromagnetic position-detection and mapping system 2 can be determined by means of the markers 6.

In the case of the present embodiment, a volume data set or a 3D image of the heart H of the patient P is acquired using the C-arm X-ray device 1. This involves rotating the C-arm 7 of the C-arm X-ray device 1, which arm is provided with an X-ray source 8 and an X-ray receiver 9, around its orbital axis O or its angulation axis A within an angle range of about 190°, a plurality of 2D projections of the heart H of the patient P being taken from different projection directions. An image calculator 10 reconstructs a volume data set or 3D-image of the heart H of the patient P from said 2D X-ray projections. As the heart H is an active organ, the reconstruction of the volume data set or of the 3D-image of the heart H is achieved in the case of the present embodiment by using ECG signals from the patient P recorded during the acquisition of the 2D X-ray projections. The ECG equipment is not shown in FIG. 1 since it is designed in the known manner. Such an ECG-triggered reconstruction method is described, for example, in DE 10 2005 016 472 A1.

The reconstructed 3D-image of the heart H of the patient P also shows the markers 6. In preparation for the calculation of the aforementioned coordinate transformation, the images 16 of the markers 6 are located manually or even automatically in the 3D image using a method of pattern recognition and the coordinates of the markers 6 in the coordinate system CB assigned to the 3D image or to the coordinate system CR assigned to the C-arm X-ray device 1 are determined. This is possible because the dimensions and geometries of the C-arm X-ray device 1 and the projection geometries for the acquisition of the 2D X-ray projections are known. Here the coordinate origin of the coordinate system CB can be located, for example, in the isocenter IZ of the C-arm 7. As stated in the aforementioned, the markers 6 are arranged on the patient P such that they do not conceal any interesting structures of the heart H. Finally, the volume data set or 3D-image of the heart H of the patient P is stored in an intermediate image memory 11 so that the 3D image data can be used for merging or superimposition with other data or for a navigation of the catheter 3 or of another instrument.

In the case of the present embodiment, the electromagnetic position-detection and mapping system 2 comprises three transmitters 21 combined in one unit 20, each of which generates an electromagnetic alternating field, said alternating fields differing from each other. In the case of the present embodiment, the unit 20 that comprises the three transmitters 21 is detachably arranged in a defined position on the patient supporting device 5. If the unit 20 for example, were to cause interference during the recording of the 2D X-ray projections, it can then be removed from the patient supporting device 5 and be arranged on the patient supporting device 5 once again after the X-ray projections have been taken. The unit 20 with the three transmitters 21 could also be detachably arranged in a different location on the patient supporting device 5 or on the C-arm X-ray device 1 that is arranged in a defined manner relative to the patient supporting device 5, as indicated, for example, with dotted lines in FIG. 1 on the C-arm 7 of the C-arm X-ray device 1.

The catheter 3 of the electromagnetic position-detection and mapping system 2 is provided at the catheter tip with three sensors that are not further shown in FIG. 1. If the catheter 3 is moved within the alternating fields of the transmitters 21, the position of the catheter 3 can be determined in the coordinate system CM assigned to the electromagnetic position-detection and mapping system 2 using a computing device 23 of the electromagnetic position-detection and mapping system 2.

In the case of the present embodiment, the patient P has a reference sensor 22 of the electromagnetic position-detection and mapping system 2 fitted to their back, such that movements of the patient P can also be determined using the electromagnetic position-detection and mapping system. A patient coordinate system CP is assigned to the reference sensor 22 or to the patient P.

For the determination of the coordinate transformation between the coordinate system CR assigned to the C-arm X-ray device 1 or the coordinate system CB assigned to the image acquired using the C-arm X-ray device 1 and the coordinate system CM assigned to the electromagnetic position-detection and mapping system or to the patient coordinate system CP, the coordinates of the markers 6 in the coordinate system CM assigned to the electromagnetic position-detection and mapping system 2 or to the patient coordinate system CP are determined by the markers being touched by the catheter 3. Preferably, but not necessarily, the coordinates of the markers 6 are shown in the patient coordinate system CP.

If the coordinates of the markers 6 are available in the image calculator 10 in the coordinate system CB assigned to the volume data set or to the 3D image, and likewise if the coordinates of the markers 6 are available in the computing device 23 of the electromagnetic position-detection and mapping system 2 in the patient coordinate system CP or in the coordinate system CM assigned to the electromagnetic position-detection and mapping system 2, the respective coordinate transformations can be determined using the image computer 10, the computing device 23 or a further computing device 24.

Furthermore, before or during a catheter application on the heart H of the patient P, electroanatomical mapping data, preferably 3D mapping data, can be acquired using the catheter 3. By means of electromagnetic sensors incorporated into the tip of the catheter 3, it is possible to measure the changes in voltage induced by catheter movements of the catheter 3 within the alternating fields of the transmitters and to measure the position of the catheter 3 at any time with the aid of mathematical algorithms. Through point-by-point mapping of areas of a chamber of the heart with the catheter 3 and simultaneous detection of the electrical signals from the sensors, an electroanatomical three-dimensional map or 3D mapping data is or are thus produced, it being possible for the electrical signals to be reproduced in a color-coded manner, for example. Since the coordinate transformation between the coordinate system CB assigned to the 3D image produced by the C-arm X-ray device 1 and the patient coordinate system CP or the coordinate system CM that is assigned to the electromagnetic position-detection and mapping system 2 is known, the image data for the 3D image and the 3D mapping data can be merged together or superimposed on one another and displayed on the monitor 4 in order to visually assist a catheter application on the heart H of the patient P. In addition, the catheter 3, for example, can be navigated relative to the heart H of the patient P, on the basis, for example, of the merged data, by means of an image of the catheter 3, based on the known coordinate transformation, being accordingly merged into the data that has been merged together or superimposed on one another.

It therefore becomes clear that, by means of the procedure according to the invention, it is possible for image data that has been recorded before a catheter application, using image data generated using an imaging device, to be merged or superimposed with 3D mapping data likewise generated before the catheter application or during the catheter application and a catheter 3 can be navigated using the merged or superimposed data relating to a patient P.

In order to avoid having to determine the coordinate transformation anew following a change in the position of the patient P, after the acquisition of the 3D image, the patient P, as already stated, is provided with a reference sensor 22 of the electromagnetic position-detection and mapping system 2, such that changes in the position of the patient P are determined by detection of the reference sensor 22 and the coordinate transformation can be modified according to the movement of the patient P, for example.

In order to correctly take into account a change in the position of the patient P, one method of operation allows for time synchronization to be achieved between the acquisition of the 3D image using the C-arm X-ray device 1 and the position of the patient P, by assigning the time of acquisition t1 of the 3D image to the 3D image that has been recorded, and determining the position of the patient over time, every second, for example, so that the position of the patient P at the time t1 of acquisition of the 3D image can be determined by a manual or automatic comparison of the times. In this way, it is therefore possible for a change in the position of the patient P after the acquisition of the 3D image to be detected and for this to be taken into account in transformation calculations, in particular in the merging or superimposition of image data relating to the 3D image and currently recorded mapping data or in the current navigation of the catheter 3 on the basis of the 3D image. For the time synchronization, preferably both the image calculator 10 and the computing device 23 have a clock, said clocks being synchronized with each other, via the computer 24, for example.

The position of the patient P during the acquisition of the 3D image can also be captured such that the C-arm X-ray device 1 or the image computer 10 assigns an identifier to the 3D image at the time of acquisition of the 3D image and transmits the identifier in real time at the time of acquisition of the 3D image to the electromagnetic position-detection and mapping system 2 or to the computing device 23 of the electromagnetic position-detection and mapping system 2. Said computing device 23 of the electromagnetic position-detection and mapping system 2 stores the position of the patient (P), including the identifier at the time of acquisition of the 3D image.

Furthermore, the C-arm X-ray device 1 or the image calculator 10 can retrieve the position of the patient P from the computing device 23 of the electromagnetic position-detection and mapping system 2 at the time of acquisition of the 3D image of the patient P and assign it to the 3D image that has been acquired.

In all these cases, during the acquisition of the 3D image, the position of the patient P is assigned to the 3D image of the patient P that has been acquired, so that even in the event of a change in the position of the patient P relative to the position that the patient P assumed during the acquisition of the 3D image, the transformation relationship, once determined, does not have to be determined again but can be adjusted accordingly according to the change in the patient's position. The merger or superimposition of currently acquired mapping data with image data for the 3D image acquired before the change in position or the blending in of current images of the catheter 3 into the image data for the 3D image acquired before the change in position is then achieved on the basis of the transformation relationship that has been modified according to the new position of the patient P.

Should the transmitter unit 20 have to be removed from the patient supporting device 5 during the acquisition of the volume data set or of the 3D image using the C-arm X-ray device, due to a lack of space, for example, then the position of the patient P can be determined before the removal and after the re-connection of the transmitter unit 20. If the patient P has moved during the acquisition of the volume data set or of the 3D image, then it is for the user to decide, depending on the position values acquired, whether the position value recorded before the removal of the transmitter unit 20, the position value recorded after the re-connection of the transmitter unit 20 or a mean value is assigned to the volume data set or to the 3D image as a position value. In this procedure, too, the position of the patient P is determined during the acquisition of the volume data set or of the 3D image.

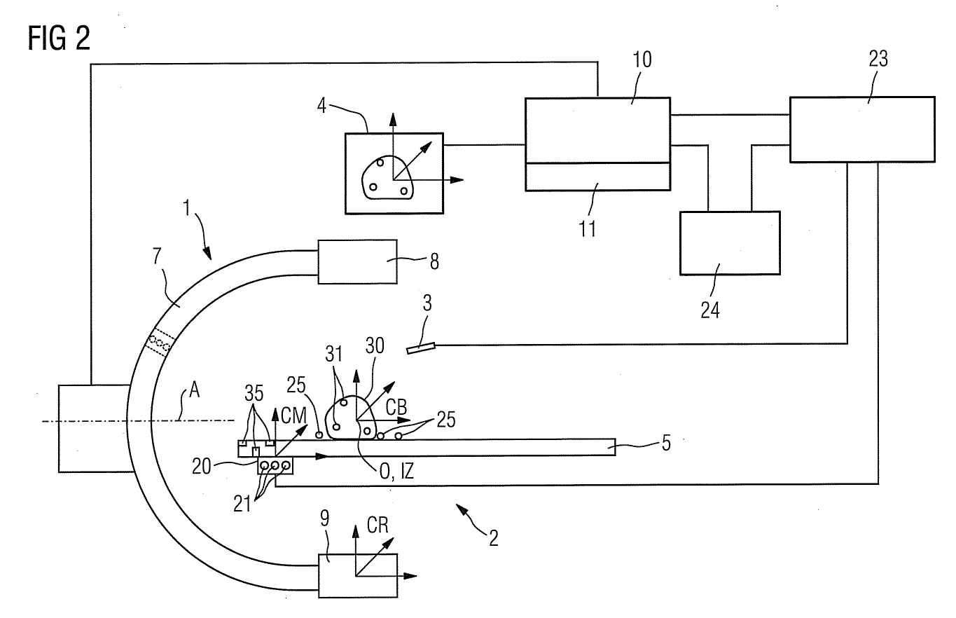

The determination of the coordinate transformation between the coordinate system CR assigned to the C-arm X-ray device 1 or the coordinate system (CB) that is assigned to the 3D image generated by the C-arm X-ray device 1 and the coordinate system CM assigned to the electromagnetic position-detection and mapping system 2 or to the patient coordinate system CP, wherein markers 6 are arranged on the patient CP, has been described with the aid of FIG. 1. The determination of the coordinate transformation can also be carried out in what is known as an offline procedure, that is, without the patient P. This can be achieved as shown in FIG. 2, for example, such that preferably three X-ray positive sensors 25 of the electromagnetic position-detection and mapping system 2, for example, three catheters 3, are arranged on the patient supporting device 5 and the coordinates of the position sensors 25 in the coordinate system CM assigned to the electromagnetic position-detection and mapping system 2 are determined. Moreover, at least two projection images from different projection directions or an X-ray image of the position sensors 25 are acquired by the C-arm X-ray device 1, the images of the position sensors 25 are located in the images or in the 3D image either manually or using a method of pattern recognition, and the coordinates of the position sensors are determined in the coordinate system CR assigned to the C-arm X-ray device 1 or in the coordinate system CB assigned to the projection images or to the 3D image. If the coordinate transformation is determined on the basis of coordinates that have been determined in the coordinate systems, the C-arm X-ray device 1 and the electromagnetic position-detection and mapping system 2 are calibrated relative to each other.

Alternatively, a phantom 30 provided with X-ray positive markers 31 can be used. If a 3D image of the phantom 30, for example, in which the X-ray positive markers 31 are depicted is generated by the C-arm X-ray device 1, then the images of the markers 31 can be located in the 3D image again either manually by clicking on them by hand or automatically using a method of pattern recognition. On this basis, based on the known design of the C-arm X-ray device and the known projection geometries of the 2D projections that form the basis of the 3D image, the coordinates of the markers 31 in the coordinate system CR assigned to the C-arm X-ray device 1 or in the coordinate system CB assigned to the 3D image can be determined. The determination of the coordinates of the markers 31 in the coordinate system CM assigned to the electromagnetic position-detection and mapping system 2 is achieved, for example, by touching the markers 31 with the catheter 3. The coordinate transformation can then be calculated again using the coordinates determined in the coordinate systems.

Alternatively, all the markers 31 of the phantom 30 can be position sensors in the electromagnetic position-detection and mapping system 2, such that to determine the coordinates of the markers 31 in the coordinate system CM of the electromagnetic position-detection and mapping system 2, the markers 31 do not have to be expressly touched by the catheter 3.

Alternatively, only one marker 31 of the phantom 30 can be a position sensor of the electromagnetic position-detection and mapping system 2. In this case, if the positions of the remaining markers 31 of the phantom 30 relative to the at least one position sensor of the phantom 30 are known, then only the coordinates of this one position sensor in the coordinate system CM of the electromagnetic position-detection and mapping system 2 have to be determined, while the others can be determined from their known positions in relation to the position sensor of the phantom 30 that has been detected.

A further possible way of establishing a relationship between the coordinate system CR assigned to the C-arm X-ray device 1 and the coordinate system CM assigned to the electromagnetic position-detection and mapping system 2 consists in arranging, for example, three positioning and orientation sensors 35 of the electromagnetic position-detection and mapping system 2 in a defined manner on or in the patient supporting device 5. In this case, on the basis of the known design relationship between the C-arm X-ray device 1 and the patient supporting device 5 and the known design arrangement of the three positioning and orientation sensors 35 on or in the patient supporting device 5, the transformation relationship between the coordinate system CR assigned to the C-arm X-ray device 1 and thus also between the coordinate system CB assigned to an image recorded using the C-arm X-ray device 1 and the coordinate system CM assigned to the electromagnetic position-detection and mapping system 2 can be determined by means of the detection of the three positioning and orientation sensors 35 detectable with the electromagnetic position-detection and mapping system 2. In this case, the three positioning and orientation sensors 35 of the electromagnetic position-detection and mapping system 2 can be designed to be detachable from the patient supporting device by, for example, arranging said sensors in one or a plurality of detachable modules.

The C-arm X-ray device 1 and the electromagnetic position-detection and mapping system 2 can thus be calibrated relative to each other offline. Consequently, patient images can then be taken using the C-arm X-ray device 1 and the electromagnetic position-detection and mapping system 2 and image data acquired using the C-arm X-ray device 1 and mapping data from the electromagnetic position-detection and mapping system 2 can be merged together or superimposed on one another. Furthermore, images of navigation instruments such as the catheter 3 that are detectable using the electromagnetic position-detection and mapping system 2 can be blended into the images acquired using the C-arm X-ray device 1 and/or into the mapping data and/or into data that have been merged together or superimposed on one another. This allows changes in the position of the patient P to be registered, determined, and taken into account accordingly through the detection of the reference sensor 22, as already disclosed.

Moreover, the markers arranged on the patient can also be positioning and orientation sensors in the electromagnetic position-detection and mapping system.

Furthermore, a plurality of reference sensors can be arranged on the patient P.

As already disclosed, apart from the catheter 3, other instruments provided with a positioning and orientation sensor can also be used in the device disclosed. If, for example, instead of or in addition to the catheter 3, a catheter is used with which image data from inside the body can be acquired, for example, the ultrasound catheter AcuNav from Siemens Medical Solutions, then the image data acquired using this catheter can be combined with image data for the 3D image acquired using the image generation device. Catheter monitoring systems such as the Niobe System from Stereotaxis can also be used in the device.

1.-56. (canceled)

57. A method for visually assisting a catheter application on a heart of a patient, comprising:
acquiring an image of the patient by a C-arm X-ray device;
acquiring electroanatomical mapping data of the patient by an electromagnetic position-detection and mapping system;
determining a coordinate transformation between a coordinate system of the C-arm x-ray device and a coordinate system of the electromagnetic position-detection and mapping system;
calibrating the C-arm X-ray device and the electromagnetic position-detection and mapping system in relation to each other based on the coordinate transformation;
determining a position of the patient during the acquisition of the image and the acquisition of the electroanatomical mapping data; and
assigning the position of the patient to the image and the electroanatomical mapping data.

58. The method as claimed in claim 57,
wherein a patient supporting device is assigned to the C-arm X-ray device,
wherein the electromagnetic position-detection and mapping system comprises a catheter with a sensor and a transmitter for generating an electromagnetic alternating field,
wherein the transmitter is arranged on the C-arm X-ray device or on the patient supporting device and is detachable from the C-arm X-ray device or from the patient supporting device,
wherein the transmitter is arranged on the C-arm of the C-arm X-ray device, and
wherein a positioning and orientation sensor of the electromagnetic position-detection and mapping system is arranged on or in the patient supporting device and is detachable from the patient supporting device.

59. The method as claimed in claim 57, wherein the coordinate transformation is determined by a marker that is detectable by the electromagnetic position-detection and mapping system.

60. The method as claimed in claim 59, wherein the marker is an X-ray positive marker and an image of the marker is depicted in at least two X-ray projections taken from different projection angles or in a 3D image.

61. The method as claimed in claim 60, wherein the image of the marker is located manually or automatically in the X-ray projections or in the 3D image by a method of pattern recognition.

62. The method as claimed in claim 59, wherein the marker is touched by a position sensor of the electromagnetic position-detection and mapping system.

63. The method as claimed in claim 59, wherein the marker is an X-ray positive position sensor of the electromagnetic position-detection and mapping system.

64. The method as claimed in claim 59, wherein a phantom comprising the marker is provided and the marker of the phantom is a position sensor of the electromagnetic position-detection and mapping system.

65. The method as claimed in claim 64, wherein the phantom comprises a further marker and a position of the further marker is known relative to the position sensor.

66. The method as claimed in claim 64, wherein a coordinate transformation between a coordinate system of the phantom and a coordinate system of the electromagnetic position-detection and mapping system is known.

67. The method as claimed in claim 59, wherein the marker is arranged on the patient.

68. The method as claimed in claim 57, wherein image data of the image is merged or superimposed with the electroanatomical mapping data taking into account the position of the patient.

69. The method as claimed in claim 57, wherein a part of a catheter that is detectable by the electromagnetic position-detection and mapping system is blended into the image of the patient or the electroanatomical mapping data taking into account the position of the patient.

70. The method as claimed in claim 57, wherein a reference sensor of the electromagnetic position-detection and mapping system is arranged on the patient for determining the position of the patient.

71. The method as claimed in claim 70, wherein the position of the patient or the coordinate system of the C-arm x-ray device and the coordinate system of the electromagnetic position-detection and mapping system is determined relative to the reference sensor.

72. The method as claimed in claim 57, wherein the position of the patient at a time of the acquisition of the image is determined by comparing a time of the determination of the position of the patient and the time of the acquisition of the image.

73. The method as claimed in claim 57,
wherein an identifier is assigned to the image of the patient at a time of the acquisition of the image and transmitted to the electromagnetic position-detection and mapping system, and
wherein the electromagnetic position-detection and mapping system stores the position of the patient and the identifier at the time of the acquisition of the image.

74. The method as claimed in claim 57, wherein the position of the patient is retrieved from the electromagnetic position-detection and mapping system at a time of the acquisition of the image of the patient and assigned to the image of the patient.

75. The method as claimed in claim 57, wherein the position of the patient is taken into account in merging or superimposing image data of an image of the patient acquired before the catheter application with the electroanatomical mapping data recorded before or during the catheter application or in a navigation of a catheter.

76. A device for visually assisting a catheter application on a heart of a patient, comprising:
a C-arm X-ray device that acquires an image of the patient;
an electromagnetic position-detection and mapping system that acquires electroanatomical mapping data of the patient; and
a computing device that:
determines a coordinate transformation between a coordinate system of the C-arm x-ray device and a coordinate system of the electromagnetic position-detection and mapping system,
calibrates the C-arm X-ray device and the electromagnetic position-detection and mapping system in relation to each other based on the coordinate transformation,
determines a position of the patient during the acquisition of the image and the acquisition of the electroanatomical mapping data; and
assigns the position of the patient to the image and the electroanatomical mapping data.
